(19) United States
(12) Patent Application Publication
(10) Pub. No.: US 2025/0241717 A1
(43) Pub. Date: Jul. 31, 2025

(54) WIRELESS ARCHITECTURES FOR SURGICAL ROBOTIC SYSTEMS

(71) Applicant: Covidien LP, Mansfield, MA (US)

(72) Inventors: William J. Peine, Boston, MA (US); Burt D. Ochs, Methuen, MA (US)

(21) Appl. No.: 18/853,767

(22) PCT Filed: Jan. 19, 2023
(86) PCT No.: PCT/US2023/011126

§ 371 Date: Oct. 3, 2024

Related U.S. Application Data
(60) Provisional application No. 63/339,541, filed on May 9, 2022.

(51) Int. Cl. A61B 34/30; A61B 17/00; A61B 34/20; G06N 20/00

(52) U.S. Cl. CPC A61B 34/30; A61B 17/00; G06N 20/00; A61B 2017/00221; A61B 2034/2055; A61B 2034/2059; A61B 2034/305

(57) ABSTRACT

A surgical robotic system includes a control tower and a robotic system component. The control tower includes a tower Li-Fi transceiver configured to communicate on a first communication data channel. The robotic system component includes a robotic system component Li-Fi transceiver configured to communicate first data, and a self-aligning mechanism. The self aligning mechanism includes an imaging device configured to capture images configured to allow detection of a detected a geospatial location of the tower Li-Fi transceiver, and an actuatable base configured for aligning the imaging device with the detected geospatial location of the tower Li-Fi transceiver. The robotic system component Li-Fi transceiver is mounted to the actuatable base.

### CROSS-REFERENCE TO RELATED APPLICATIONS

The present application claims the benefit of and priority to U.S. Provisional Patent Application Ser. No. 63/339,541, filed on May 9, 2022, the entire content of which being hereby incorporated by reference.

### BACKGROUND

Surgical robotic systems may include a surgeon console controlling one or more surgical robotic arms, each including a surgical instrument having an end effector (e.g., forceps or grasping instrument). In operation, the robotic arm is moved to a position over a patient and the surgical instrument is guided into a small incision via a surgical access port or a natural orifice of a patient to position the end effector at a worksite within the patient's body. The surgeon console includes hand controllers which translate user input into movement of the surgical instrument and/or end effector.

Wireless communications in surgical robotic systems are beneficial to reduce the complexity of setup and minimize the need for cables draped across the operating room floor. A challenge of this approach is that wireless transmissions are susceptible to interference and intermittent loss of information packets. This can lead to unintended conditions and motions during teleoperated control as well as temporary loss of motion of the instruments within the patient.

### SUMMARY

According to one embodiment of the present disclosure, a surgical robotic system is disclosed. The surgical robotic system includes a robotic arm, a control tower and a surgeon console. The robotic arm includes a first arm transceiver including a first communication data channel configured for communicating first data and a second arm transceiver including a second communication data channel configured for communicating second data. The surgeon console includes a first console transceiver configured to communicate on the first communication data channel and a second console transceiver configured to communicate on the second communication data channel. The control tower includes a first tower transceiver configured to communicate on the first communication data channel and a second tower transceiver configured to communicate on the second communication data channel. The first communication data channel and the second communication data channel are wireless communication data channels. The surgical robotic system is configured to switch from communicating using one of the first communication data channel or the second communication data channel.

In accordance with aspects of the disclosure, the first communication data channel uses a different communication technology than the second communication data channel. The communication technology may include at least one of Wi-Fi, 5G, Li-Fi, or Bluetooth

In an aspect of the present disclosure, the first communication data channel may use a different frequency than the second communication data channel.

In another aspect of the present disclosure, the control tower may further include a processor and a memory. The memory includes instructions stored thereon, which when executed by the processor, may cause the surgical robotic system to monitor channel quality of the first communication data channel and the second communication data channel and alternate communication data channels based on observed channel quality.

In yet another aspect of the present disclosure, the instructions when executed by the processor may further cause the surgical robotic system to determine if there is data loss based on the monitored channel quality. The first communication data channel may communicate at a first data-rate and the second communication data channel may communicate at a second data-rate. The second data-rate may be lower than the first data-rate. In a case where data loss is detected on the first communication data channel, the instructions when executed by the processor, may further cause the surgical robotic system to switch the second communication data channel to a data-rate higher than the first data-rate.

In a further aspect of the present disclosure, the first data and the second data may include a data integrity check.

In yet a further aspect of the present disclosure, the first data on the first communication data channel may be different than the second data on the second communication data channel. The data integrity check may include performing a transformation on the first data of the first communication data channel and on the second data of the second communication data channel.

In accordance with aspects of the disclosure, the first data of the first communication data channel includes data indicating a desired location of an end-effector of the robotic arm in Cartesian coordinates relative to a base frame of the robotic arm. The second data of the second communication data channel may include data indicating desired joint angles of the robotic arm. The transformation includes transforming the first data of the first communication data channel into the second data of the second communication data channel based on performing a forward and reverse kinematic algorithm, and/or transforming the second data of the second communication data channel into the first data of the first communication data channel based on performing a forward and reverse kinematic algorithm.

According to one embodiment of the present disclosure, a surgical robotic system is disclosed. The surgical robotic system includes a control tower and a robotic system component. The control tower includes a first tower transceiver including a first communication data channel configured to communicate first data and a second tower transceiver including a second communication data channel configured to communicate second data. The robotic system component includes a first robotic system transceiver configured to communicate on the first communication data channel and a second robotic system transceiver configured to communicate on the second communication data channel. The first communication data channel and the second communication data channel are wireless communication data channels. The first communication data channel and the second communication data channel are configured for simultaneous communication.

In an aspect of the present disclosure, the robotic system component may include a robotic arm and/or a surgeon console.

In another aspect of the present disclosure, the first communication data channel may use a different communication technology than the second communication data channel The communication technology may include at least one of Wi-Fi, 5G, Li-Fi, or Bluetooth.

In yet another aspect of the present disclosure, the first communication data channel may use a different frequency than the second communication data channel.

In a further aspect of the present disclosure, the control tower may further include a processor and a memory. The memory may include instructions stored thereon which, when executed by the processor, may cause the surgical robotic system to monitor channel quality of the first communication data channel and the second communication data channel, and to alternate communication data channels based on observed channel quality.

In yet a further aspect of the present disclosure, the instructions when executed by the processor, may further cause the surgical robotic system to determine if there is data loss based on the monitored channel quality. The first communication data channel may communicate at a first data-rate and the second communication data channel may communicate at a second data-rate. The second data-rate may be lower than the first data-rate. In a case where data loss is detected on the first communication data channel, the instructions when executed by the processor, may further cause the surgical robotic system to switch the second communication data channel to a data-rate higher than the first data-rate.

In accordance with aspects of the disclosure, the first data and the second data may include a data integrity check.

In an aspect of the present disclosure, the data integrity check may include comparing the first data from the first communication data channel to the second data from the second communication data channel.

In another aspect of the present disclosure, the first data on the first communication data channel may be different than the second data on the second communication data channel. The data integrity check may include performing a transformation on the first data of the first communication data channel and on the second data of the second communication data channel.

In yet another aspect of the present disclosure, the robotic system component may include a robotic arm. The first data of the first communication data channel may include data indicating a desired location of an end-effector of the robotic arm in Cartesian coordinates relative to a base frame of the robotic arm. The second data of the second communication data channel includes data indicating desired joint angles of the robotic arm. The transformation may include at least one of transforming the first data of the first communication data channel into the second data of the second communication data channel based on performing a forward and reverse kinematic algorithm, or transforming the second data of the second communication data channel into the first data of the first communication data channel based on performing a forward and reverse kinematic algorithm.

According to one embodiment of the present disclosure, a computer-implemented method for redundant wireless communications for a surgical robotic system is disclosed. The computer-implemented method includes: communicating first data between a control tower and robotic system component on a first communication data channel, simultaneously communicating second data between the control tower and the robotic system component on a second communication data channel; monitoring channel quality of the first communication data channel and the second communication data channel; and alternating communication data channels based on observed channel quality. The first communication data channel and the second communication data channel are wireless communication data channels. The robotic system component includes a robotic arm and/or a surgeon console.

According to one embodiment of the present disclosure, a surgical robotic system includes a control tower and a robotic system component. The control tower includes a tower Li-Fi transceiver configured to communicate on a first communication data channel. The robotic system component includes a robotic system component Li-Fi transceiver including a first communication data channel configured for communicating first data and a self-aligning mechanism. The self-aligning mechanism includes an imaging device configured to capture images including a detected a geospatial location of the tower Li-Fi transceiver, and an actuatable base configured to align the imaging device with the detected geospatial location of the tower Li-Fi transceiver. The robotic system component Li-Fi transceiver is mounted to the actuatable base.

In an aspect of the present disclosure, the self-aligning mechanism may further include a processor and a memory. The memory may include instructions stored thereon which, when executed by the processor, may cause the self-aligning mechanism to capture an image including the tower Li-Fi transceiver, determine a geospatial location of the tower Li-Fi transceiver based on the image, and position the robotic system component Li-Fi transceiver to align with the detected geospatial location of the tower Li-Fi transceiver.

In another aspect of the present disclosure, the geospatial location of the tower Li-Fi transceiver may be determined based on providing the image to a trained machine learning network. The image may include depth information. The geospatial location of the tower Li-Fi transceiver may be further determined based on classifying the tower Li-Fi transceiver in the image and determining the geospatial location of the tower Li-Fi transceiver based on the classification and the depth information.

In yet another aspect of the present disclosure, the self-aligning mechanism may further include a processor and a memory. The memory may include instructions stored thereon which, when executed by the processor, may cause the self-aligning mechanism to: capture an image, determine there is no tower Li-Fi transceiver within the image, and rotate and/or tilt the imaging device to search for the tower Li-Fi transceiver. The determining may be performed by a machine learning network.

In a further aspect of the present disclosure, the robotic system component may further include a wireless transceiver. In a case that no tower Li-Fi transceiver is within the image, the instructions when executed by the processor may further cause the robotic system component to establish wireless communication with the control tower using the wireless transceiver. The wireless communication may be on a second communication data channel.

In an aspect of the present disclosure, the second communication data channel may use a different communication technology than the first communication data channel. The communication technology of the second communication data channel may include infrared, visible light, ultraviolet light, Wi-Fi, 5G, or Bluetooth.

In another aspect of the present disclosure, the control tower may further include a processor and a memory. The memory may include instructions stored thereon which, when executed by the processor, may cause the surgical robotic system to monitor channel quality of the first communication data channel and the second communication data channel and select one of the first communication data channel or the second communication data channel based on the monitored channel quality.

In yet another aspect of the present disclosure, the instructions when executed by the processor, may further cause the surgical robotic system to determine if there is data loss based on the monitored channel quality. The first communication data channel communicates at a first data-rate and the second communication data channel communicates at a second data-rate. The second data-rate may be lower than the first data-rate. In response to a data loss in the first communication data channel, the instructions when executed by the processor, may further cause the surgical robotic system to switch the second communication data channel to a data-rate higher than the first data-rate.

In a further aspect of the present disclosure, the first communication data channel and the second communication data channel may include a data integrity check.

In an aspect of the present disclosure, the robotic system component may include at least one of a console or a robotic arm.

According to one embodiment of the present disclosure, a computer-implemented method for wireless communications for a surgical robotic system includes capturing an image including a tower Li-Fi transceiver of a control tower. The image is captured by an imaging device of a robotic system component. The imaging device is disposed on an actuatable base of a self-aligning mechanism. The method further includes determining a geospatial location of the tower Li-Fi transceiver based on the captured image and actuating the actuatable base to position a robotic system component Li-Fi transceiver to align with the determined geospatial location of the tower Li-Fi transceiver.

In another aspect of the present disclosure, the geospatial location of the tower Li-Fi transceiver may be determined based on providing the image to a trained machine learning network. The image includes depth information. The geospatial location of the tower Li-Fi transceiver may further be determined based on classifying the tower Li-Fi transceiver in the image and determining the geospatial location of the tower Li-Fi transceiver based on the classification and the depth information.

In yet another aspect of the present disclosure, the robotic system component may include at least one of a console or a robotic arm.

According to one embodiment of the present disclosure, a computer-implemented method for wireless communications for a surgical robotic system is presented. The method includes: capturing an image by an imaging device of a robotic system component, where the imaging device is disposed on an actuatable base of a self-aligning mechanism; and determining there is no tower Li-Fi transceiver within the image. The determining may be performed by a machine learning network.

In a further aspect of the present disclosure, the method may further include actuating the actuatable base to rotate and/or tilt a robotic system component Li-Fi transceiver to search for the tower Li-Fi transceiver of a control tower.

In an aspect of the present disclosure, the robotic system component may further include a wireless transceiver. In a case that no tower Li-Fi transceiver is with the image, the method may further include causing the robotic system component to establish wireless communication with the control tower using the wireless transceiver. The wireless communication may be on a second communication data channel.

In another aspect of the present disclosure, the tower Li-Fi transceiver may be configured to communicate on a first communication data channel. The second communication data channel may use a different communication technology than the first communication data channel. The communication technology of the second communication data channel may include at least one of infrared, visible light, ultraviolet light, Wi-Fi, 5G, or Bluetooth.

In yet another aspect of the present disclosure, the method may further include monitoring channel quality of the first communication data channel and the second communication data channel and selecting one of the first communication data channel or the second communication data channel based on the monitored channel quality.

In a further aspect of the present disclosure, the method may further include determining if there is data loss based on the monitored channel quality. The first communication data channel may communicate at a first data-rate and the second communication data channel may communicate at a second data-rate. The second data-rate may be lower than the first data-rate. In response to a data loss in the first communication data channel, the surgical robotic system may switch the second communication data channel to a data-rate higher than the first data-rate.

In an aspect of the present disclosure, the first communication data channel and the second communication data channel may include a data integrity check.

### BRIEF DESCRIPTION OF THE DRAWINGS

Various embodiments of the present disclosure are described herein with reference to the drawings wherein:

FIG. 1 is a schematic illustration of a surgical robotic system according to an embodiment of the present disclosure including a control tower, a console, and one or more surgical robotic arms each disposed on a mobile cart;

FIG. 2 is a perspective view of a surgical robotic arm of the surgical robotic system of FIG. 1;

FIG. 3 is a perspective view of a mobile cart having a setup arm with the surgical robotic arm of the surgical robotic system of FIG. 1;

FIG. 4 is a schematic diagram of a computer architecture of the surgical robotic system of FIG. 1;

FIG. 5 is a schematic diagram of a communication network of the surgical robotic system of FIG. 1;

FIG. 6 is a schematic diagram of exemplary interfaces of a communications management subsystem suitable for use with the surgical robotic system of FIG. 1;

FIG. 7 shows a flow diagram for a method for redundant wireless communications suitable for use with the surgical robotic system of FIG. 1;

FIG. 8 is a schematic diagram of a communication network of the surgical robotic system of FIG. 1;

FIG. 9 is a flow diagram of a machine learning algorithm of the computer-controlled method for clinical workspace simulation using the system of FIG. 1;

FIG. 10 is a diagram of layers of a neural network of FIG. 9 in accordance with aspects of the disclosure; and

FIG. 11 shows a flow diagram for a method for redundant wireless communications suitable for use with the surgical robotic system of FIG. 1.

### DETAILED DESCRIPTION

Embodiments of the presently disclosed surgical robotic system are described in detail with reference to the drawings, in which like reference numerals designate identical or corresponding elements in each of the several views. As used herein the term “proximal” refers to the portion of the surgical robotic system and/or the surgical instrument coupled thereto that is closer to a base of a robot, while the term “distal” refers to the portion that is farther from the base of the robot.

As will be described in detail below, the present disclosure is directed to systems and methods for redundant wireless communications for a surgical robotic system, which includes a surgeon console, a control tower, and one or more mobile carts having a surgical robotic arm coupled to a setup arm. The surgeon console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command. The disclosed systems and methods enable redundancy in the communications to detect and compensate for lost packets and corrupted data without interrupting teleoperated control.

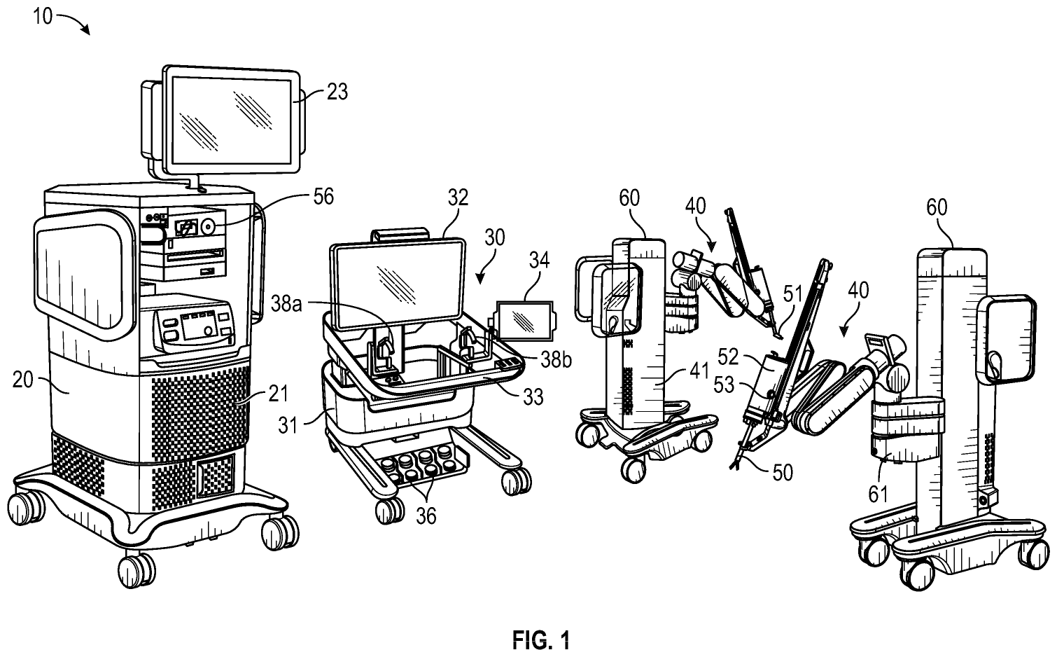

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 removably coupled thereto. The robotic arms 40 also couple to the movable cart 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first display 32, which displays a video feed of the surgical site provided by camera 51 of the surgical instrument 50 disposed on the robotic arm 40, and a second display 34, which displays a user interface for controlling the surgical robotic system 10. The first and second displays 32 and 34 are touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, 6G, LiDAR, Wi-Fi6, optical, Wi-Fi, Li-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

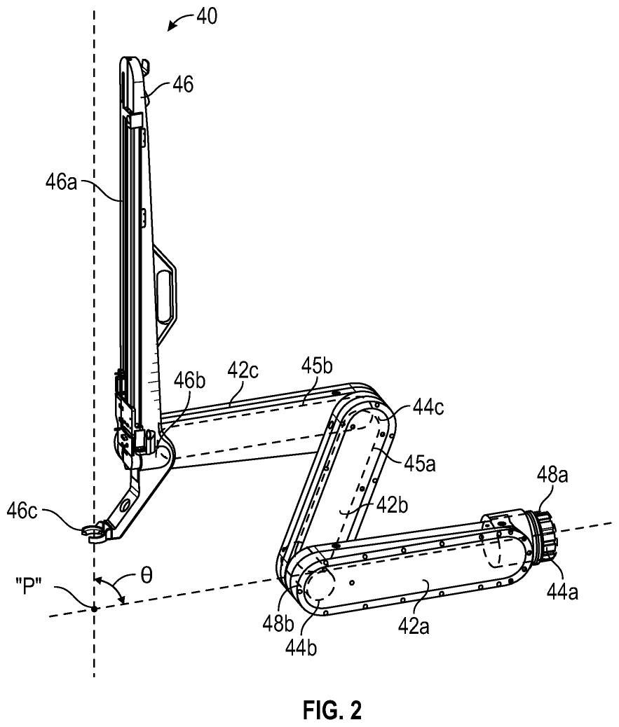

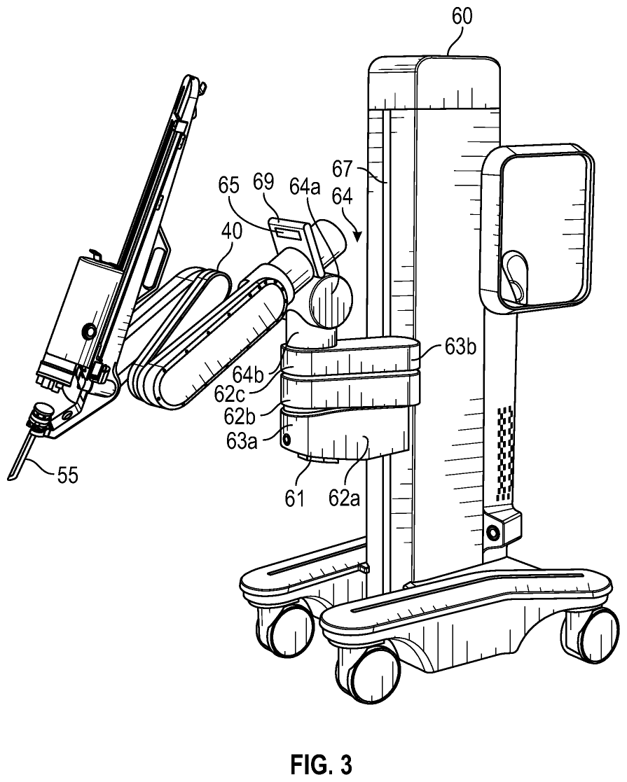

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

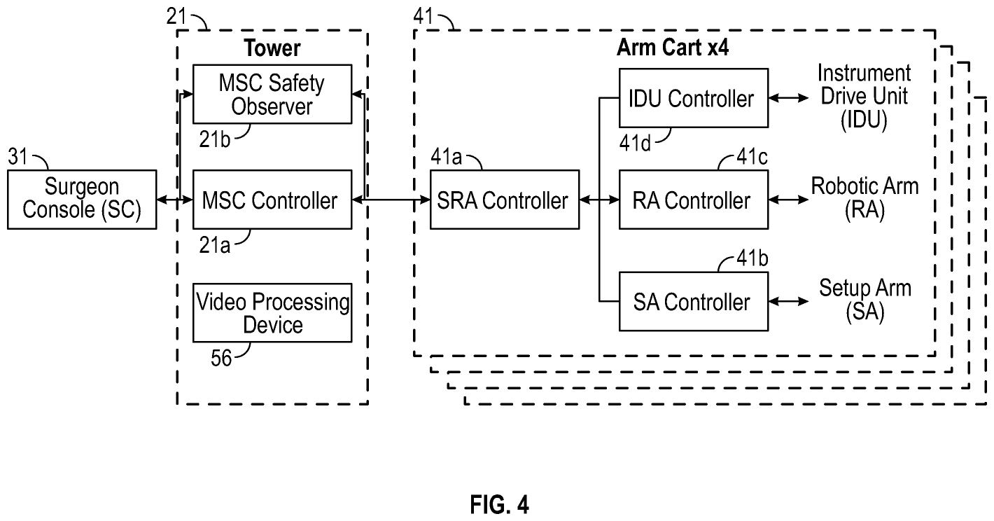

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

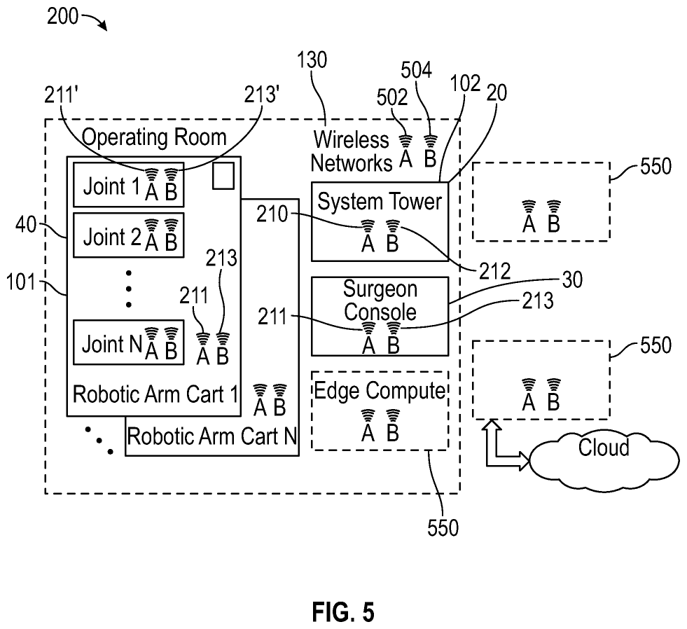

FIG. 5 illustrates components and interfaces of a wireless communication system 200 for use with the surgical robotic system 10. The wireless communication system 200 is embedded or otherwise included in the control tower 20 and robotic system component (e.g., console 30 and/or robotic arm 40) and includes a wireless communications network 130.

The control tower 20 includes a plurality of transceivers, e.g., a first tower transceiver 210 and a second tower transceiver 212. While only two communication channels are described herein, it is envisioned that any number (e.g., 3, 4, 5, or more) of channels may be provided to enable redundant communication. The first tower transceiver 210 includes a first communication data channel 502 configured for communicating first data, and a second tower transceiver 212 includes a second communication data channel 504 configured for communicating second data. In embodiments, multiple wired interfaces may be used along with or in lieu of the redundant wireless interfaces.

The robotic system component 535 includes a first robotic system transceiver 211 configured to communicate on the first communication data channel 502, and a second robotic system transceiver 213 configured to communicate on the second communication data channel 504.

The control tower 20 also includes a wireless controller 102, configured to invoke a wireless mode by activating and establishing communications with wireless device 101. In the situation or mode where a physical cable or wire is not present, or where the user chooses to operate in the wireless mode, the wireless controller 102 may invoke a wireless mode by activating and establishing communications with the wireless device 101. In the wireless mode, a wireless communications network 130 enables the exchange of control signals, data, and information between the wireless controller 102 and the wireless device 101.

In this mode, the wireless controller 102 initiates a wireless device-searching mode to locate and pair with an available wireless device 101 to establish a primary wireless communications path across the wireless communications network 130. The wireless controller 102 searches for a unique wireless device 101 using, for example, Bluetooth™ short-range radio techniques. Searching is complete when the correct wireless device 101 is located. At this point, the wireless controller 102 ‘pairs up’ or ‘matches’ with the unique wireless device 101 to enable communication of control signal and other device information, such as battery condition. The specific techniques and details associated with Bluetooth™ searching and “pairing” mechanism are generally known to those skilled in the art. Alternate searching and locating techniques may be employed depending on the transmission protocol employed. For example, 802.11g may employ link control procedures known to those skilled in the art and specified by the standard, while a protocol such as infrared line-of-sight communications may employ optical locating and searching techniques again known to those skilled in the art. Subsequently, the wireless controller 102 establishes one or more backup wireless communication paths in a similar manner over the wireless communications network 130. The master controller 208 embedded within the wireless controller 102 establishes a primary connection through the first tower transceiver 210 and establishes a backup connection through the second tower transceiver 212. If more than one backup communication path is present and available, the master controller also establishes these communication paths as additional backup connections between 214 and 215 and so forth.

Alternating between channels ensures that two copies of the same data stream are transmitted to the wireless device 101 within the wireless device 101. Moreover, the master controller 208 may continuously monitor the channel quality of all active paths. Monitoring the channel quality may include measuring signal strength, signal quality, or checking data integrity and observing other relevant parameters to determine current path connection condition and reporting the measured result to the wireless controller 102. The wireless device 101 may report additional observed non-fixed device management information, including but not limited to current battery charge condition, not pertaining to communications path integrity through the communications network 130 to the wireless controller 102. In addition, the communications path channel quality observed by the wireless device 101 may be presented to the user. If either the primary or backup data communications path becomes disconnected or degraded during use, a visual alert, an audible alert, and any combination thereof may be provided to the user. In embodiments, the visual alert may be realized by blinking an LED when either path becomes disconnected, wherein a constantly lit LED may indicate that both communications paths are connected and available for use. Similarly, a periodic audible alert may be sounded when either communications path becomes disconnected.

The term “wireless device” or “wireless medical device” or “non-fixed wireless device” or the like as used herein means a device capable of receiving and/or transmitting information wirelessly, i.e., over the air, using either a radio, light wave (e.g., infrared, visible light, and/or ultraviolet light) or other communication technique that does not require a physical connection, such as a wire. Wireless devices that may realize the reception and transmission of data include, but are not limited to, those devices meeting or complying with the Institute of Electrical and Electronics Engineers (IEEE) 802.11 (Wi-Fi), Ericson Bluetooth™ specifications for short-range radio technology, an Infrared Data Association (IrDA) light wave techniques, and/or 5G.

As shown in FIG. 5, the system 10 includes a wireless communication system 200 for both a first and a second communication data channel. Individual components of the robotic system 10 (control tower 20, surgeon console 30, arm carts 60, robotic arms 40, etc.) and even individual actuators 64a, 64b, 64c, (FIG. 3) may receive and transmit information on these communication data channels. The communication can also extend beyond the room to the hospital and then on to the cloud.

Data may be transferred in many protocols in the form of packets of data, but other data transfer formats may be employed. Packets may contain fields such as headers and lower-level protocol information embedded in the packet. Data is transferred via packets using certain common protocols. In an alternate embodiment, communications and packets could be divided between channels, such as pitch packets for robotic arm 40 movements on one channel and yaw packets for robotic arm 40 movements on the other. Such a design would enable faster data transfer, may save power, and may enable cross-checking, but failure of one channel would require relatively immediate transfer to the other channel and carrying both pitch and yaw packets in this example over the remaining channel.

The first and second communication data channels 502, 504 (FIG. 5) may use the same technology (e.g., Wi-Fi, 5G, Li-Fi, 6G, LiDAR, Wi-Fi6, or Bluetooth™), different technologies, and/or use the same or different frequency ranges. Dropped or corrupted packets are usually caused by interference at the frequency of the communication channel, so operating at different frequencies greatly decreases the likelihood that one source of interference would knock out both channels at the same time. The system may then detect a loss on either channel and use the data from the unaffected channel for robotic control. This redundancy in data transmission enables additional data integrity checks to be added for additional safety.

Each of the joints 44a, 44b, 44c, of the robotic arm 40 may include a first arm transceiver 211′ and a second arm transceiver 213′. The first arm transceiver 211′ and the second arm transceiver 213′ may be configured to control the respective actuators 64a, 64b, 64c. There are several advantages provided by the disclosed technology, including reduced wiring and a simplified mechanical design—since each smart actuator 64a, 64b, 64c in the system receives commands over wireless, the only wired connections need to be power (from a battery or wall supply) and an additional wired safety channel. Construction of the robotic arm 40 is easier since the communications wires for distal joints of the robotic arm do not have to be passed through proximal joints. By not passing the wire through proximal joints, construction is simplified, the size and weight of the robot arm 40 are reduced, and the faults caused by electrical connections failing in these sections of the arm are eliminated.

Another advantage includes a simplified electrical architecture. Since each smart actuator 64a, 64b, 64c communicates directly to the control tower 20, the only wiring required for each joint is a daisy-chained power connection and serial safety communication link. In aspects, the safety communication channel may be communicated on the same wires/cables/conductors as the power using higher frequency signals that “float” on top of the power.

Another advantage includes a simpler, more centralized software architecture. Wireless communication to each joint means that all of the high-level behavior controllers (state machines, kinematics, and robot behavior algorithms) may be located in one computation node in the control tower 20 or, for example, an edge compute processor 550 (FIG. 5). Since fewer computation nodes are involved in the system control, all the system variables and signals are available in the control tower 20, which makes data logging and debugging much easier. The software is also much easier to maintain and test since there are fewer nodes performing computation. The disclosed architecture makes it easier to deploy and upgrade the software since the software may be on a node at an edge computer tied to the cloud.

A further advantage includes a simpler and improved safety architecture. Since all of the low-level signals may be transmitted to the control tower 20, the safety systems can be more complex and determine when situations impose a safety risk, or the situation can be handled while maintaining operation of the system. This improves uptime of the system while maintaining safety.

The system 10 could communicate with each actuator individually and have a computation node in the robotic arm 40. This would enable the control tower 20 to communicate with the robotic arm 40 to control functions on the robotic surgical system 10 that are beyond the scope of individual actuators. Examples may include user interfaces, buttons, input devices, visualization systems electrosurgery generators, other camera or audio devices on the arm observing the sterile field and operating theater, and/or localization technology used to register the arms to each other and image data.

In aspects, the same principles of the wireless smart actuators described above may be applied to the input device in the surgeon console 30, or other control devices, such as wireless position sensing of the surgeon's hands. One communication point may communicate with a plurality of actuators that are physically close to each other. These actuators may be a subset of the total number of actuators on the robotic arm 40.

The wireless communication channels may be used to control or transmit data to and from other devices in the operating room (OR), such as screens, cameras, microphones, proximity sensors, AR/VR headsets, or other equipment not directly tied to the robotic system.

The wireless communication network 130 shown in FIG. 5 may be extended to include the entire surgical floor or hospital so that surgical robotic systems or components can transmit data when outside the OR. The extended wireless communication network enables localization of system components within the hospital, remote service of the components during off-hours, the transmission of use and diagnostic data to the cloud, and automatic software updates. The simplified architectures described above also facilitate these same things.

In aspects, the communication system 200 may include the ability to transmit more power on a primary data channel and less power on a secondary cross-checking or complementary channel, thereby decreasing overall power requirements or increasing power transmission on the primary channel.

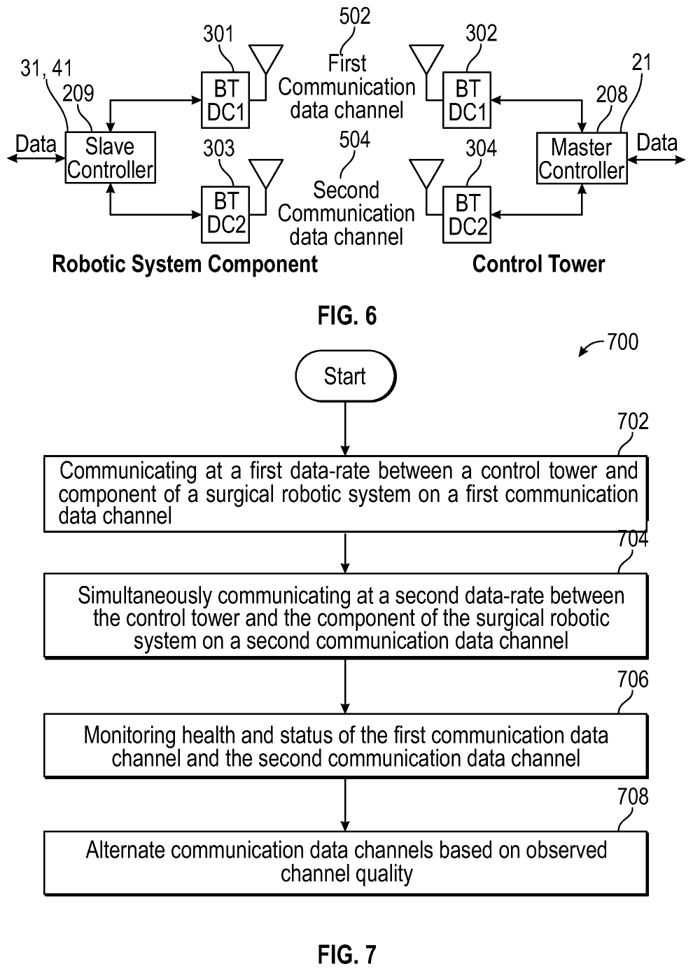

FIG. 6 shows that the wireless transmission and reception of data and information across the primary and backup data channels are realized using a peer-to-peer wireless communication protocol such as Bluetooth™ short-range radio technology. While Bluetooth™ is used as an example, other communication technologies are contemplated.

The wireless controller 102 (FIG. 5) initiates a wireless device-searching mode utilizing data channel one at 302 on the first communication data channel 502 to locate and pair with an available data channel one (DC1) at 301 to establish a primary wireless communications data channel over the wireless communications network 130. Subsequently, the communications wireless controller 102 initiates a wireless device-searching mode utilizing data channel two (DC2) at 304 on the second communication data channel 504 to locate and pair with an available Bluetooth™ data channel two (DC2) at 303 to establish a backup wireless communications data channel over the wireless communications network 130. The primary and backup data channels, as shown in FIG. 6, may provide a bi-directional redundant connection between the control tower 20 and the robotic system component 535 (30, 40). Data may now be communicated across these channels using the alternating communication technique described previously. Note that if non-bidirectional protocols are employed, one data channel may engage in one-way communication when not in active use, i.e., when the channel has failed or been turned off.

The master controller 208 and slave controller 209 may provide one or more of Cyclic Redundancy Codes (CRC) checksum validation, path control, or data validation to manage the communication of data across each data channel (i.e., primary and backup). If the master controller 208 detects that the primary data channel between points 302 and 301 is lost, corrupted, or unstable due to interference or other causes, the master controller 208 promotes the backup data channel between points 304 and 303 to become the primary data channel. The newly promoted data channel two (DC2) between points 304 and 303 continues to operate as the primary data channel until or even when the failed data channel one is restored. During this operational aspect, the slave controller 209 may observe that receiving data channel one (DC1) at 301 is no longer able to receive data transmitted by data channel one (DC1) at 302. In this situation, the slave controller 209 automatically switches to receiving data channel two (DC2) at 303 as the primary channel and continues to receive data uninterrupted as transmitted by data channel two at 304. As a result, no data interruption occurs during the surgery or procedure being performed. In a similar manner, the master controller 208 may promote the backup data channel two as primary whenever a signal quality, or any combination thereof is observed. This method of promotion continues during the surgical day to ensure reliable and high availability of the communicated data stream between the control tower 20 and for example, the robotic arm 40. Moreover, if additional backup data channels are available, the present design may promote one of these additional backup data channels to replace the failed data channel and may return the failed data channel to the backup channel pool when restored.

Referring now to FIG. 7, a flow diagram for a method 700 for redundant wireless communications for the surgical robotic system 10 of FIG. 1 is shown. In various embodiments, the operation of FIG. 7 can be performed by another type of system and/or during another type of procedure. The following description will refer to a surgical robotic system, but it will be understood that such description is exemplary and does not limit the scope and applicability of the present disclosure to other systems and procedures.

Initially, at step 702, the wireless communication system 200 communicates first data between a control tower 20 and robotic system component 535 on a first communication data channel. The robotic system component 535 may include a robotic arm 40 and/or a surgeon console 30 (FIG. 1). The first communication data channel and the second communication data channel may be wireless communication data channels. The first communication data channel may use a different communication technology (e.g., Wi-Fi, 5G, Li-Fi, and/or Bluetooth™) than the second communication data channel.

Next, at step 704, the wireless communication system 200 simultaneously communicates second data between the control tower 20 and the robotic system component 535 on a second communication data channel.

Next, at step 706, the wireless communication system 200 monitors the channel quality of the first communication data channel and the second communication data channel.

The data from the two channels can be compared against each other as a redundant check. The data may include the same data, transmitted over both channels, or it could be a formulaic derivative of the data. The redundant channel data may be a subsampling or other time-based calculation from the original full bandwidth data.

In some aspects, the same information could be transmitted on both channels. This allows for a simple replacement of lost information if one channel is corrupted, as well as a packet-by-packet comparison for subtle levels of corruption.

In aspects, different types of data may be transmitted on each channel with a known transformation between the two. For example, one channel could transmit the desired end-effector's location in Cartesian coordinates (XYZ and role-pitch-yaw angles) relative to a base frame, and the other channel could transmit all the desired joint angles of the arm. A forward and inverse kinematic algorithm may be used to transform one dataset into the other. This provides redundancy and a safety level check of the main system controller calculations.

In some aspects, the data transmitted on the two channels may communicate at different data rates. The first data communication channel could transmit at the full data rate (e.g., about 1 kHz), and the second data communication channel may, for example, transmit at a fraction of the full rate (e.g., 20, 100, 200, or 500 Hz). The second data communication channel may be used to perform a safety check when things are normal and then be switched to the higher transmission rate if corruption on the first data communication channel is detected.

In aspects, the above approach may be split into the transmit and receive directions. For example, one channel could be full bandwidth for data from the control tower 20 to the robotic arm 40, or other robotic system component 535, and then a subsampled rate for data from the robotic arm to the control tower. The second communication data channel 504 may do the opposite—downsampling for data from the tower to the robotic arm and full bandwidth from the robotic arm to the control tower. A benefit of this technology is that it may balance the data transmission levels between the two channels.

In aspects, the first communication data channel and the second communication channel may be configured for switching between a first communication protocol to a second communication protocol based on the data integrity check.

Using the safety and high reliability of the disclosed two-channel communication arrangement, several new software and computer architectures for surgical robotic systems are possible. One example of this is to have each actuator in the robotic arm communicate directly with control tower or, for example, an edge compute node, enabling a “smart actuator.” Each joint may include an amplifier, sensors, and controller for position or torque control. Additional sensors may be connected to these smart actuators and use the communication channel of the joint to transmit data to the main system controller.

Next, at step 708, the wireless communication system 200 may alternate communication data channels based on observed channel quality.

In aspects, the wireless communication system 200 may determine if there is a safety issue and trigger switching data communication channels based on the determined safety issue. The wireless communication system 200 may determine if there is a failure on both the first communication data channel 502 and second communication data channel 504 and provide a prompt to establish a wired backup connection.

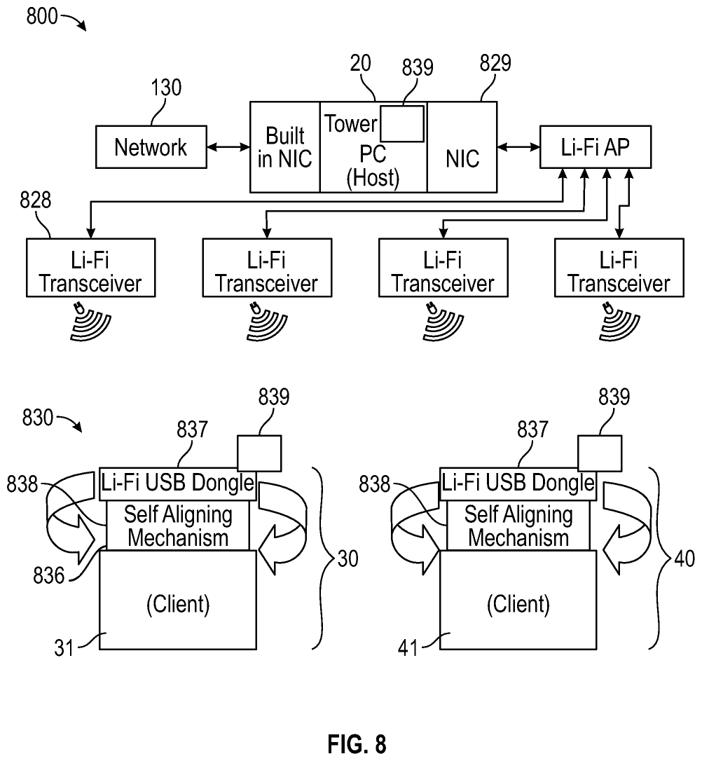

Referring to FIG. 8, illustrates components and interfaces of a light-based wireless communication system 800 for use with the surgical robotic system 10. The wireless communication system 800 may work with the wireless communication system 200 of FIG. 5. The wireless communication system 800 is configured for coexistence with other communication devices which use, for example, Wi-Fi, Bluetooth™, cellular and other RF-based systems in the hospital ecosystem. The wireless communication system 800 is embedded or otherwise included in both the control tower 20 and a robotic system component 830 (e.g., console 30 and/or robotic arm 40). The wireless communication system 800 includes a wireless communications network 130.

The wireless communication system 800 provides the benefit of improving bedside access and setup by eliminating cables. The wireless communication system 800 ensures a secure, reliable, low-latency connection for the robotic surgical system 10.

The control tower 20 includes one or more Li-Fi transceivers 828. “Li-Fi” is a wireless communication technology that utilizes light to transmit data and position between devices. Li-Fi is capable of transmitting data at high speeds over visible light, ultraviolet, and infrared spectrums. The Li-Fi transceiver(s) 828 may be connected to the control tower 20 via a wired or wireless network. For example, the control tower 20 may include a network interface card (NIC) 829 configured for wired network communications with one or more Li-Fi transceivers 828. The Li-Fi transceiver(s) 828 may be located at the control tower 20 or remotely located in various positions in the OR. For example, a first tower transceiver 828 may include a first Li-Fi communication data channel 502 (FIG. 5) configured for communicating first data, and a second tower Li-Fi transceiver 828 may include a second communication data channel 504 configured for communicating second data (FIG. 5). In embodiments, multiple wired interfaces may be used along with or in lieu of the redundant wireless interfaces.

The robotic system component 830 (e.g., console 30 and/or robotic arm 40) generally includes a Li-Fi transceiver 837 and a vision-based self-aligning mechanism 838. The vision-based self-aligning mechanism 838 is configured to determine a geospatial location of a tower Li-Fi transceiver 828 and rotate and/or tilt the Li-Fi transceiver 837 of the robotic system component 830 to align with a detected geospatial location of the tower Li-Fi transceiver 828. The vision-based self-aligning mechanism 838 includes an actuatable base 836 configured for mounting the imaging device 839 and the robotic system component Li-Fi transceiver 837. The actuatable base 836 is rotatably and tiltably connected to the robotic system component 830. The actuatable base 836 is configured to be rotated and/or tilted under the control of a computer 21, 31, 41.

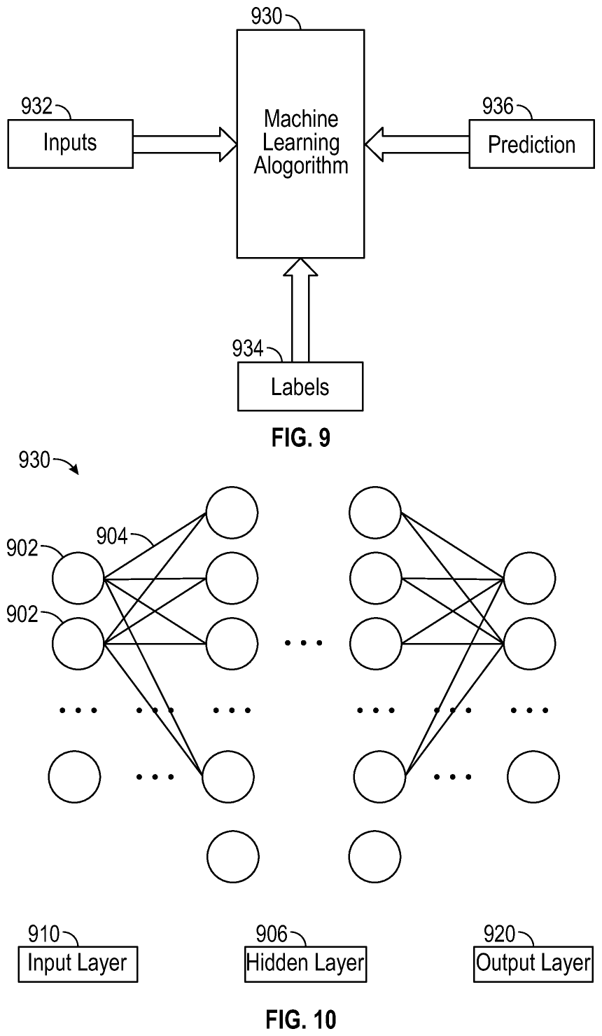

The vision-based self-aligning mechanism 838 may include an imaging device 839 configured to capture images for processing by a computer 21, 31, 41. In aspects, the imaging device 839 may be located on the control tower 20. In aspects, the imaging device 839 may include a stereoscopic imaging device configured to capture depth information. In aspects, the computer 21, 31, 41, may use a machine learning network 930 (FIG. 9) to determine a geospatial location of the tower Li-Fi transceiver 828. In aspects, the vision-based self-aligning mechanism 838 may use external depth cameras to align multiple Li-Fi transceivers. The wireless communication system 800 provides the benefit of using multiple transceivers 828 and vision-based self-alignment mechanisms 838 to overcome line-of-site constraints.

With reference to FIG. 9, the controller 209 may include the machine learning network 930 configured to make the evaluations described herein. For example, the controller 209 may use machine learning to classify (prediction 936) images supplied as an input 932 to the machine learning network 930. Machine learning may include a convolutional neural network (CNN) and/or a support vector machine (SVM). The CNN may be trained on previous data, for example, images of objects such as the specific robotic surgical equipment used for an operation. Images may be labeled 934 for use in training the machine learning network 930.

Referring to FIG. 10, generally, the machine learning network 930 (e.g., a convolutional deep learning neural network) of FIG. 9 includes at least one input layer 910, a plurality of hidden layers 906, and at least one output layer 920. The input layer 910, the plurality of hidden layers 906, and the output layer 920 all include neurons 902 (e.g., nodes). The neurons 902 between the various layers are interconnected via weights 904. Each neuron 902 in the machine learning network 930 computes an output value by applying a specific function to the input values coming from the previous layer. The function that is applied to the input values is determined by a vector of weights 904 and a bias. Learning in the deep learning neural network progresses by making iterative adjustments to these biases and weights. The vector of weights 904 and the bias are called filters (e.g., kernels) and represent particular features of the input (e.g., a particular shape). The machine learning network 930 may output logits.

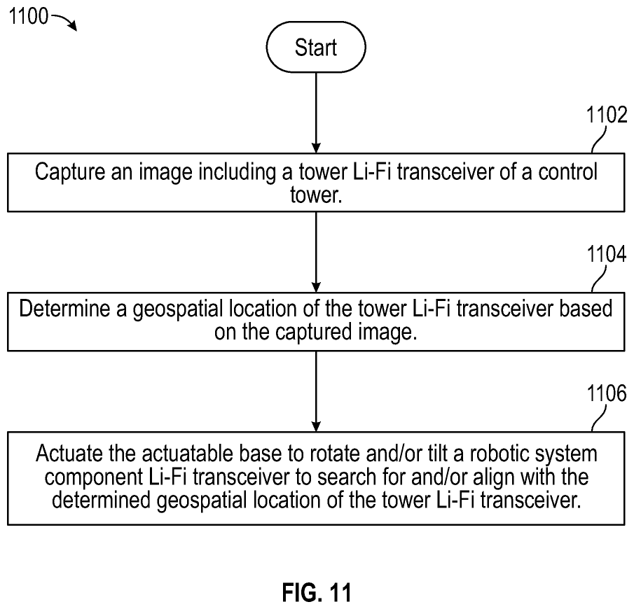

Referring now to FIG. 11, a flow diagram for a method 1100 for wireless communications for the surgical robotic system 10 of FIG. 1 is shown. In various embodiments, the operation of FIG. 11 can be performed by another type of system and/or during another type of procedure. The following description will refer to a surgical robotic system, but it will be understood that such description is exemplary and does not limit the scope and applicability of the present disclosure to other systems and procedures.

Initially, at step 1102, an imaging device 839 of a robotic system component 830 captures an image including a tower Li-Fi transceiver 828 of a control tower 20. The robotic system component 830 may include a robotic arm 40 and/or a surgeon console 30 (FIG. 1). The imaging device 839 may be disposed on an actuatable base 836 of a self-aligning mechanism 838 (FIG. 8). The imaging device 839 may include a stereoscopic imaging device, configured to capture depth information.

Next, at step 1104, the wireless communication system 800 determines a geospatial location of the tower Li-Fi transceiver 828 based on the captured image.

In aspects, the geospatial location of the tower Li-Fi transceiver 828 may be determined based on providing the image to a trained machine learning network 930 (FIG. 9). The image may include depth information. The geospatial location of the tower Li-Fi transceiver 828 may be further determined based on classifying the tower Li-Fi transceiver 828 in the image and/or determining the geospatial location of the tower Li-Fi transceiver 828 based on the classification and the depth information.

The wireless communication system 800 may determine there is no tower Li-Fi transceiver within the image and rotate and/or tilt the imaging device to search for the tower Li-Fi transceiver 828.

In aspects, the robotic system component 830 may further include a wireless transceiver (e.g., robotic system transceiver 212), and wherein in a case that no tower Li-Fi transceiver 828 is within the image, the robotic system component 830 may establish wireless communication with the control tower 20 using the wireless transceiver.

Li-Fi communication may be on a first communication data channel. The wireless communication may be on a second communication data channel. In aspects, the wireless communication system 800 may monitor the channel quality of the first communication data channel and the second communication data channel and select one of the first communication data channel or the second communication data channel based on the monitored channel quality.

In aspects, when the wireless communication system 800 is communicating via the wireless transceiver, the wireless communication system 800 may perform a search for the tower Li-Fi transceiver 828. If the tower Li-Fi transceiver 828 is found, the Li-Fi communication connection may be reestablished, and the wireless communication system 800 will switch back to Li-Fi communication.

Next, at step 1106, the wireless communication system 200 actuates the actuatable base 836 to position (e.g., rotate and/or tilt) a robotic system component Li-Fi transceiver 828 to align with the determined geospatial location of the tower Li-Fi transceiver 828.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a control tower including a tower Li-Fi transceiver configured to communicate on a first communication data channel; and
a robotic system component including:
a robotic system component Li-Fi transceiver including a first communication data channel configured for communicating first data; and
a self-aligning mechanism including:
an imaging device configured for capturing images configured to allow detection of a detected a geospatial location of the tower Li-Fi transceiver; and
an actuatable base configured for aligning the imaging device with the detected geospatial location of the tower Li-Fi transceiver, wherein the robotic system component Li-Fi transceiver is mounted to the actuatable base.

2. The surgical robotic system according to claim 1, wherein the self-aligning mechanism further includes:
a processor; and
a memory, including instructions stored thereon which, when executed by the processor, cause the self-aligning mechanism to:
capture an image including the tower Li-Fi transceiver;
determine a geospatial location of the tower Li-Fi transceiver based on the image; and
position the robotic system component Li-Fi transceiver to align with a detected geospatial location of the tower Li-Fi transceiver.

3. The surgical robotic system according to claim 2, wherein the geospatial location of the tower Li-Fi transceiver is determined based on:
providing the image to a trained machine learning network, wherein the image includes depth information;
classifying the tower Li-Fi transceiver in the image; and
determining the geospatial location of the tower Li-Fi transceiver based on the classification and the depth information.

4. The surgical robotic system according to claim 1, wherein the self-aligning mechanism further includes:
a processor; and
a memory, including instructions stored thereon which, when executed by the processor, cause the self-aligning mechanism to:
capture an image;
determine there is no tower Li-Fi transceiver within the image, wherein the determining is performed by a machine learning network; and
rotate and/or tilt the imaging device to search for the tower Li-Fi transceiver.

5. The surgical robotic system according to claim 4, wherein the robotic system component further includes a wireless transceiver, and wherein in a case that no tower Li-Fi transceiver is within the image, the instructions when executed by the processor cause the robotic system component to establish wireless communication with the control tower using the wireless transceiver, wherein the wireless communication is on a second communication data channel.

6. The surgical robotic system according to claim 5, wherein the second communication data channel uses a different communication technology than the first communication data channel, wherein the communication technology of the second communication data channel includes at least one of infrared, visible light, ultraviolet light, Wi-Fi, 5G, or Bluetooth.

7. The surgical robotic system according to claim 6, wherein the control tower further includes:
a processor; and
a memory, including instructions stored thereon, which when executed by the processor, cause the surgical robotic system to:
monitor channel quality of the first communication data channel and the second communication data channel; and
select one of the first communication data channel or the second communication data channel based on the monitored channel quality.

8. The surgical robotic system according to claim 7, wherein the instructions when executed by the processor further cause the surgical robotic system to determine if there is data loss based on the monitored channel quality,
wherein the first communication data channel communicates at a first data-rate and the second communication data channel communicates at a second data-rate, wherein the second data-rate is lower than the first data-rate, and
wherein in response to a data loss in the first communication data channel, the instructions when executed by the processor further cause the surgical robotic system to switch the second communication data channel to a data-rate higher than the first data-rate.

9. The surgical robotic system according to claim 8, wherein the first communication data channel and the second communication data channel include a data integrity check.

10. The surgical robotic system according to claim 1, wherein the robotic system component includes at least one of a console or a robotic arm.

11. A computer-implemented method for wireless communications for a surgical robotic system comprising:
capturing an image including a tower Li-Fi transceiver of a control tower, wherein the image is captured by an imaging device of a robotic system component, and wherein the imaging device is disposed on an actuatable base of a self-aligning mechanism;
determining a geospatial location of the tower Li-Fi transceiver based on the captured image; and
actuating the actuatable base to position a robotic system component Li-Fi transceiver to align with the determined geospatial location of the tower Li-Fi transceiver.

12. The computer-implemented method according to claim 11, wherein the geospatial location of the tower Li-Fi transceiver is determined based on:
providing the image to a trained machine learning network, wherein the image includes depth information;
classifying the tower Li-Fi transceiver in the image; and
determining the geospatial location of the tower Li-Fi transceiver based on the classification and the depth information.

13. The computer-implemented method according to claim 11, wherein the robotic system component includes at least one of a console or a robotic arm.

14. A computer-implemented method for wireless communications for a surgical robotic system comprising:
capturing an image by an imaging device of a robotic system component, wherein the imaging device is disposed on an actuatable base of a self-aligning mechanism; and
determining there is no tower Li-Fi transceiver within the image,
wherein the determining is performed by a machine learning network.

15. The computer-implemented method according to claim 14, further comprising actuating the actuatable base to move a robotic system component Li-Fi transceiver to search for the tower Li-Fi transceiver of a control tower.

16. The computer-implemented method according to claim 15, wherein the robotic system component further includes a wireless transceiver, and wherein in a case that no tower Li-Fi transceiver is with the image, the method further includes causing the robotic system component to establish wireless communication with the control tower using the wireless transceiver, wherein the wireless communication is on a second communication data channel.

17. The computer-implemented method according to claim 16, wherein the tower Li-Fi transceiver is configured to communicate on a first communication data channel, wherein the second communication data channel uses a different communication technology than the first communication data channel, and wherein the communication technology of the second communication data channel includes at least one of infrared, visible light, ultraviolet light, Wi-Fi, 5G, or Bluetooth.

18. The computer-implemented method according to claim 17, further comprising:
monitoring channel quality of the first communication data channel and the second communication data channel; and
selecting one of the first communication data channel or the second communication data channel based on the monitored channel quality.

19. The computer-implemented method according to claim 18, further comprising:
determining if there is data loss based on the monitored channel quality,
wherein the first communication data channel communicates at a first data-rate and the second communication data channel communicates at a second data-rate, wherein the second data-rate is lower than the first data-rate, and
wherein in response to a data loss in the first communication data channel, the surgical robotic system switches the second communication data channel to a data-rate higher than the first data-rate.

20. The computer-implemented method according to claim 19, wherein the first communication data channel and the second communication data channel include a data integrity check.
